(19) United States
(12) Patent Application Publication
(10) Pub. No.: US 2015/0290120 A1
(43) Pub. Date: Oct. 15, 2015

(54) SKIN CARE COMPOSITION COMPRISING PLANT EXTRACTS

(71) Applicant: CGTN C.V., WILLEMSTAD (CW)

(72) Inventor: Sreedhar R. Nalabolu, Irving, TX (US)

(73) Assignee: CGTN C.V., WILLEMSTAD (CW)

(21) Appl. No.: 14/683,797

(22) Filed: Apr. 10, 2015

Related U.S. Application Data
(60) Provisional application No. 61/978,138, filed on Apr. 10, 2014.
(60) Provisional application No. 61/978,785, filed on Apr. 11, 2014.

(51) Int. Cl. A61K 8/97; A61Q 19/08; A61Q 19/00; A61K 9/00; A61K 8/49

(52) U.S. Cl. CPC A61K 8/97; A61K 9/0014; A61K 8/498; A61Q 19/007; A61Q 19/08; A61K 2800/591

(57) ABSTRACT

Exemplary skin care compositions and methods of using such compositions are provided. In one embodiment, a topical composition includes extract of bidens pilosa, centella asiatica leaf cell extract, and a dermatologically acceptable carrier.

### CROSS-REFERENCE TO RELATED APPLICATIONS

This application claims priority to U.S. Provisional Patent Application No. 61/978,138 filed Apr. 10, 2014, which is incorporated herein by reference in its entirety and U.S. Provisional Patent Application No. 61/978,785 filed Apr. 11, 2014, which is incorporated herein by reference in its entirety.

### TECHNICAL FIELD

This disclosure relates in general to topical skin care compositions and methods for application to human skin and, in particular, but not by way of limitation, to skin care composition comprising plant extracts for improving the condition and appearance of skin.

### BACKGROUND OF THE DISCLOSURE

Skin is subject to deterioration through dermatological disorders, environmental abuse (wind, air conditioning, central heating) or through the normal aging process (chronoaging) which may be accelerated by exposure of skin to sun (photoaging). In recent years the demand for cosmetic compositions and cosmetic methods for improving the appearance and condition of skin has grown enormously.

Consumers are increasingly seeking “anti-aging” cosmetic products which treat or delay the visible signs of chronoaging and photoaging skin such as wrinkles, lines, sagging, hyperpigmentation and age spots.

Consumers also frequently seek other benefits from cosmetic products in addition to anti-aging. The concept of “sensitive skin” has also raised the consumer demand for cosmetic products which improve the appearance and condition of sensitive, dry, rough and/or flaky skin and to soothe red, irritated and/or itchy skin. Consumers also desire cosmetic products which treat spots, pimples, blemishes etc.

### SUMMARY

In a first embodiment, there is provided a topical composition including (a) extract of bidens pilosa; (b) centella asiatica leaf cell extract; and (c) a dermatologically acceptable carrier.

In certain aspects, the topical composition further includes mauritia flexuosa fruit oil.

In certain aspects, the topical composition further includes elaeis guineessis oil.

In certain aspects, the topical composition further includes gossypium herbaceum seed oil.

In certain aspects, the topical composition further includes linum usitatissimum seed oil.

In certain aspects, the topical composition further includes tocopherol.

In certain aspects, the topical composition further includes aloe barbadensis leaf juice, natural albumen, octyldodecyl olivate, hydrolyzed quinoa, and tocopherol.

In certain aspects, the topical composition further includes jojoba esters, camellia sinensis leaf extract, hydrogenated lecithin, ubiquinone, bisabolol, terapeptide-30, and palmitoyl dipeptide-5 diaminobutyroyl hydroxythreonine.

In a second embodiment, a method for treating an area of skin to reduce DNA fragmentation induced by ultra-violet radiation comprises topically applying a composition to the area of skin. The composition includes extract of bidens pilosa and centella asiatica leaf cell extract.

In certain aspects the composition further comprises mauritia flexuosa fruit oil.

In certain aspects the composition further comprises aloe barbadensis leaf juice, natural albumen, octyldodecyl olivate, hydrolyzed quinoa, and tocopherol.

In certain aspects the composition further comprises oryza sativa bran oil, montanov L, montanov 68, stearic acid, glycerin, glyceryl stearate, ricinus communis seed oil, cetyl alcohol, olus oil, chondrus crispus powder, sodium isostearoyl lactylate, sodium PCA, polysorbate-20, dermsoft LP, natural cucumber, and esseachem.

In certain aspects the composition further comprises jojoba esters, camellia sinensis leaf extract, hydrogenated lecithin, ubiquinone, bisabolol, terapeptide-30, and palmitoyl dipeptide-5 diaminobutyroyl hydroxythreonine.

In certain aspects the composition further comprises aloe vera juice.

In certain aspects the method further includes cleansing the area of the skin prior to applying the composition.

In certain aspects the method further includes applying the composition once daily in the morning.

In certain aspects the method further includes applying the composition once daily in the evening.

In certain aspects the method further includes applying the composition once in the morning and once in the evening.

In certain aspects the method further includes applying the composition as needed for moisturizing the area of skin.

In certain aspects the method is used to treat the area of skin by reducing appearance of fine lines and wrinkles, discoloration, uneven texture, enlarged pores, aging or loose skin.

In a third embodiment, a topical composition includes (a) extract of bidens pilosa; (b) mauritia flexuosa fruit oil; (c) jojoba esters; (d) centella asiatica leaf cell extract; and (e) cucumis sativus fruit extract.

In a fourth embodiment, a topical composition includes (a) extract of bidens pilosa; (b) mauritia flexuosa fruit oil; (c) jojoba esters; (d) centella asiatica leaf cell extract; and (e) tetrahexyldecyl ascorbate.

Other aspects, features, and advantages will become apparent from the following detailed description when taken in conjunction with the accompanying drawings, which are a part of this disclosure and which illustrate, by way of example, principles of the inventions disclosed.

### DESCRIPTION OF THE FIGURES

The accompanying figures/appendix facilitate an understanding of the various embodiments.

FIG. 1 is a table according to one illustrative embodiment of a complex that may be used in part, in total or as part of a composition described herein.

FIG. 2A-B is a table according to one illustrative embodiment of a composition that may be used either in the morning or night, but preferably at night.

FIG. 3A-B is a table according to one illustrative embodiment of a composition that may be used either in the morning or night, but preferably at night.

FIG. 4A-C is a table according to one illustrative embodiment of a composition that may be used either in the morning or night, but preferably in the morning.

FIG. 5A-C is a table according to one illustrative embodiment of a composition that may be used either in the morning or night, but preferably in the morning.

### DETAILED DESCRIPTION

The compositions, formulas, compounds as described herein may be used in a variety of personal care and skin care products. The compositions may be in a variety of forms, including, but not limited to an emulsion, lotion, milk, liquid, solid, cream, gel, mousse, ointment, paste, serum, stick, spray, tonic, aerosol, foam, etc.

In all embodiments, of the present invention, all percentages are by weight of the total composition, unless specifically stated otherwise. All ratios are weight ratios, unless specifically stated otherwise. All ranges are includes and combinable. The number of significant digits conveys neither a limitation on the indicated amounts nor on the accuracy of the measurements.

The term “vitamin A” is a generic description for all C20-β-ionone derivatives, which qualitatively exhibits the biological activity of transretinol. Vitamin A (retinol) and its derivatives, whether they are natural or synthetic, are referred to as retinoids (RET) and may have significant impacts on vertebrates organisms, including embryogenesis, reproduction, vision, regulation of inflammation, growth and cellular, normal, and neoplastic differentiation.

From a biological stand point, RET takes its effect by binding to specific nuclear receptors, known as RARs (Retinoic Acid Receptors) and RXRs (Retinoids Receptors), which belong to the family of transcriptional regulators induced by binders. RET function by crossing the cellular membrane into the cytoplasm, binding to the cytosolic proteins denominated CRBP or CRABP (Cytosolic Retinol Bound Protein or Cytosolic Retinoic Acid Bound Protein), and then forming a complex that allows its passage into the cell nucleus. Once inside the nucleus, RET binds to the receptors RAR or RXR, forming a complex operable in binding to RARE (Responsive Element Retinoic Acid). RARE are part of the DNA and represents the promoter region of the target gene, thus, controlling its transcription. Besides this mechanism of action, RET acts in an indirect way without binding to the DNA.

RETs are among the most prescribed and recommended agents in dermatologic therapy. Benefits such as cellular regeneration, exfoliation, and collagen synthesis indicate the importance of RETs in the prevention and treatment of several skin imperfections. Biological effects of RET may include improvement of fine lines, of acne, increased smoothness, improvement of actinic keratose, reduction of hyperpigmentation, among others.

However, there are side effects and other problems in using classic retinoids, and much research has taken place by the cosmetic industry to find alternative, active ingredient to the retinoids that are commonly used in conventional dermatology.

“Skin-Conditioning Agents” as used herein means cosmetic ingredients used to create special effects on skin. This group includes substances believed to enhance the appearance of dry or damaged skin and substantive materials which adhere to the skin to reduce flaking and restore suppleness.

“Dermatologically acceptable,” as used herein, means that the compositions or components described are suitable for use in contact with human keratinous tissue without undue toxicity, incompatibility, instability, allergic response, and the like.

“Effective amount” as used herein means an amount of a compound or composition sufficient to significantly induce a positive benefit, preferably a positive keratinous tissue appearance or feel benefit, including independently or in combination the benefits disclosed herein, but low enough to avoid serious side effects (i.e., to provide a reasonable benefit to risk ratio, within the scope of sound judgment of the skilled artisan).

“Improving skin condition” as used herein means effecting a visually and/or tactilely perceptible positive change, or benefit, in skin appearance and feel. Benefits that may be provided include, but are not limited to, one or more of the following: Reducing the appearance of wrinkles and coarse deep lines, fine lines, crevices, bumps, and large pores; thickening of keratinous tissue; skin lightening; preventing loss of skin elasticity, for example, due to loss, damage and/or inactivation of functional skin elastin, resulting in such conditions as elastosis, sagging, loss of skin recoil from deformation; change in coloration to the skin, for example, under-eye circles, blotchiness (e.g., uneven red coloration due to, for example, rosacea), sallowness, discoloration caused by hyperpigmentation, etc.

“Signs of skin aging,” as used herein, include but are not limited to, all outward visibly and tactilely perceptible manifestations, as well as any macro- or microeffects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; loss of skin elasticity; discoloration (including undereye circles); blotchiness; sallowness; hyperpigmented skin regions such as age spots and freckles; collagen breakdown.

“Hyperpigmentation,” as used herein, refers to an area of skin wherein the pigmentation is greater than that of an adjacent area of skin (e.g., a pigment spot, an age spot, and the like).

“Derivatives,” as used herein, includes but is not limited to, amide, ether, ester, amino, carboxyl, acetyl, and/or alcohol derivatives of a given compound.

“Isomers,” as used herein, is understood to include D-isomers, L-isomers, and/or DL-isomers of a given compound.

“Salts,” as used herein, includes but is not limited to sodium, potassium, calcium, ammonium, manganese, copper, and/or magnesium salts of a given compound.

“Extract,” as used herein, means material that has been extracted from a plant.

“Antioxidants” as used herein means ingredients employed in cosmetics to prevent or retard product spoilage from rancidity (or deterioration from reaction with oxygen). Antioxidants play a vital role in maintaining the quality, integrity, and safety of cosmetic products. Typical cosmetic antioxidants include reducing agents and free radical scavengers.

“Film Formers” as used herein means materials which produce a continuous film on skin. These films are used in cosmetics for diverse purposes, e.g., in forming facial masks.

“Skin-Conditioning Agents—Emollient” as used herein means cosmetic ingredients which help to maintain the soft, smooth, and pliable appearance of skin. Emollients function by their ability to remain on the skin surface or in the stratum corneum to act as lubricants, to reduce flaking, and to improve the skin's appearance.

“Surfactants—Emulsifying Agents,” “Surfactants,” or “Emulsifying Agents” as used herein are employed in cosmetics to prepare emulsions. The efficacy of emulsifying agents depends on their ability to reduce surface tension, to form complex films on the surface of emulsified droplets, and to create a repulsive barrier on emulsified droplets to prevent their coalescence. Emulsion Stabilizers and Viscosity-Increasing Agents can be used as auxiliary emulsifiers to facilitate the process of emulsification or to retard physical changes in emulsions throughout their shelf-life.

“Humectants” as used herein means ingredients used in cosmetic products to retard moisture loss from the product during use. This function is generally performed by hygroscopic materials. The efficacy of humectants depends to a large extent on the ambient relative humidity. Humectants which are used for moisturizing skin may be listed separately under Skin-Conditioning Agents—Humectants.

“Skin-Conditioning Agents—Occlusive” as used herein means cosmetic ingredients which retard the evaporation of water from the skin surface. By blocking the evaporative loss of water, occlusive materials increase the water content of skin. Occlusive agents are generally lipids which tend to remain on the skin surface. Occlusive is frequently attributed to Skin-Conditioning Agents—Emollient. Skin-Conditioning Agents—Humectant and Skin-Conditioning Agents—Miscellaneous which sometimes exhibit occlusive properties upon application to the skin.

“Viscosity Increasing Agents” as used herein means and are used to thicken the aqueous portions of cosmetic products. Their ability to perform this function is related to their water solubility or hydrophilic nature. They are widely employed in various types of emulsions. Related functions include Binders, Film Formers, and Emulsion Stabilizers.

“Chelating Agents,” also called sequestrates, as used herein means ingredients that have the ability to complex with and inactivate metallic ions in order to prevent their adverse effects on the stability or appearance of cosmetic products. At times it is important to complex calcium or magnesium ions which are incompatible with a variety of cosmetic ingredients. Chelation of ions, for example iron or copper, helps retard oxidative deterioration of finished products.

“Aliphatic,” as used herein, is understood to include both saturated and unsaturated, straight chain (i.e., unbranched) and branched, acyclic, cyclic or polycyclic aliphatic hydrocarbons, which are optionally substitute with one or more functional groups. As will be appreciated by one of ordinary skill in the art, “Aliphatic,” is intended to include, but is not limited to, alkyl, alkenyl, alkynyl, cycloalkyl, cycloalkenyl and cycloalkynyl moieties. “Alkyl,” as used herein, is understood to include straight, branched and cyclic alkyl groups. Further, “Alkyl,” as used herein, is understood to include substituted and unsubstituted groups. Analogous conventions apply to other generic terms such as “alkenyl,” “alkynyl,” and the like.

“Alkenyl,” as used herein, is understood to include a monovalent group derived from a straight- or branched chain hydrocarbon moiety containing at least one carbon-carbon double bond by removal of a single hydrogen atom.

“Anti-Aging,” as used herein, is understood to include promotion of wound healing, protection against ultraviolet radiation damage (e.g., UVA and UVB rays), protection against oxidative stress and/or reduces collagenase production.

“Anti-Inflammatory,” as used herein, is understood to include protection against chemical induced pro-inflammatory cytokine release, protection against bacterial induced (e.g., toll-like receptor-2 (TLR2)) pro-inflammatory cytokine release, protection against metal induced (e.g., toll-like receptor-4 (TLR4)) pro-inflammatory cytokine release, protection against T-cell induced pro-inflammatory cytokine release, protects against ultraviolet induced pro-inflammatory cytokine release and/or reduction of COX expression.

“Anti-Acne,” as used herein, is understood to include reduction in inflammatory lesions, reduction in microcomedone counts and/or reduction in sebum levels.

“Anti-Microbial, as used herein, is understood to include protection against Propionibacterium acnes (P. acnes) and/or Streptococcus pyogenes (S. pyogenes).

“Enantiomers, as used herein, are understood to include pairs of chemical compounds whose molecular structures have a nonsuperimposable mirror-image relationship to one another.

“Esters,” as used herein, are understood to include any and all compounds formed when a carboxylic acid and an alcohol combine and release water.

“Hydrates,” as used herein, are understood to include any and all compounds containing chemically combined water in the form of H₂O molecules.

“Solvates,” as used herein, are understood to include any and all compounds formed by the aggregation of one or more solvent molecules with a solute molecule or ion.

“Polymorphs,” as used herein, are understood to include any and all crystal forms of a chemical compound that exhibit polymorphism (i.e., two or more chemically identical but crystallography distinct forms).

I. Ingredients

The compositions disclosed herein may include one or more of the following ingredients, extracts, fractions thereof, salts, isomers, and/or derivatives thereof and, but is in no way a limitation to other ingredients. Additionally, some ingredients are described by the name required by the International Nomenclature of Cosmetic Ingredients (INCI) while others are described by trade name that comprises one or more INCI termed ingredients.

In one embodiment, Aloe Barbadensis Leaf Juice may be useful as a Skin Conditioning Agents. Non-limiting, alternatives to Aloe Barbadensis Leaf Juice are various concentrations of aloe. Aloe Barbadensis Leaf Juice may be beneficial to provide hydration. To enrich the hydrating effects, the formulations are created with the pure juice from the leaf of the Aloe Vera Plant. Aloe Vera may provide extra moisture and hydration over other formulations that use water instead of, or as a substitute to, Aloe Vera. Aloe Vera is known to provide many natural moisturizing properties helping to improve the skin's natural firmness, while at the same time helping to keep the skin hydrated. In certain aspects, a composition may include between about 55% to about 85% of Aloe Barbadensis Leaf Juice. In other aspects, a composition may include about 60% of Aloe Barbadensis Leaf Juice. In other aspects, a composition may include about 75% of Aloe Barbadensis Leaf Juice.

In one embodiment, Glycerin may be useful as Skin Conditioning Agents, a Humectant and/or a skin protectant.

In one embodiment, Dicaprylyl Carbonate may be useful as Skin Conditioning Agents and/or an Emollient.

In one embodiment, Caprylic/Capric Triglyceride may be useful as Skin Conditioning Agents and may further be Occlusive.

In one embodiment, Plumeria Auctifolia Flower Extract may be useful as Skin Conditioning Agents, having other beneficial properties.

In one embodiment, Glyceryl Stearate and/or PEG-100 Stearate may be used as Skin Conditioning Agents—Emollient, and/or Emulsifying Agents.

In one embodiment, Cetyl Alcohol may be used as Emulsion Stabilizers, Emulsifying Agents, and/or viscosity increasing agent.

In one embodiment, Jojoba Esters may be used as Skin Conditioning Agents and/or an Emollient. This botanical ingredient may be used for its moisturizing and softening effects. Light and non-oily it may add skin conditioning properties to the formulation. Even though Jojoba is referred to as oil it is actually not oil, but is a pure natural moisturizer. It contains many good nutrients for the skin such as antioxidants, Vitamin E, and essential fatty acids.

In one embodiment, Tetrahexyldecyl Ascorbate may be used as an Antioxidant and/or a Skin Conditioning Agent, having other beneficial properties.

In one embodiment, Sodium Hyaluronate may be used as a Skin Conditioning Agent and/or Moisturizing Agents.

In one embodiment, Sorbitol may be used as a Humectant and/or a Skin Conditioning Agent.

In one embodiment, Bisabolol may be used as a Skin Conditioning Agent, having other beneficial properties. Bisabolol extract, as an active botanical ingredient, has long been referred to as a very soothing agent. Often called German Chamomile, it is used in crémes and gels throughout the cosmetic industry. Many times it is chosen because it shows signs of helping to help with puffiness.

In one embodiment, Leuconostoc or Radish Root Ferment Filtrate may be used as an Antimicrobial Agent and/or a Skin Conditioning Agent.

In one embodiment, Lonicera Caprifolium (Honeysuckle) Flower Extract may be used as a Skin Conditioning Agent, having other beneficial properties.

In one embodiment, Lonicera Japonica (Honeysuckle) Flower Extract may be used as a Skin Conditioning Agent, having other beneficial properties.

In one embodiment, Populus Tremuloides Bark Extract may be used as a Skin Conditioning Agent, having other beneficial properties.

In one embodiment, Gluconolactone may be used as an Anti-acne Agent, Chelating Agent, Skin-Conditioning Agent and may have other beneficial properties.

In one embodiment, Avena Sativa (Oat) Kernel Extract may be used as an antioxidant, Skin Conditioning Agent, Emollient and may have other beneficial properties.

In one embodiment, Camellia Sinensis (Green Tea) Leaf Extract may be used as a Skin-Conditioning Agents and having other beneficial properties. Camellia Sinensis (Green Tea) Leaf Extract includes polyphenols. Polyphenols have shown powerful antioxidant activity. When used in cosmetics it is indicated tea extracts can help and assist with the prevention of collagen breakdown and help improve elasticity—an important factor in maintaining youthful and healthy looking skin.

In one embodiment, Santalum Album (Sandalwood) Extract, Phellodendron Amurense (Bark) Extract and Hordeum Distichon (Barley) Extract may be used as Skin-Conditioning Agents and having other beneficial properties.

In one embodiment, Terapeptide-30 may be used as a Skin Protectant and/or a Skin-Conditioning Agents and having other beneficial properties. Terapeptide-30 is peptides that may be selected to topically help with improving the appearance and complexion of healthy skin. Age, environmental stress and harsh conditions often leave uneven skin tones, discoloration and less than youthful skin. This peptide is believed to encourage the skin in maintaining its' own natural vitality.

In one embodiment, Palmitoyl Dipeptide-5 Diaminobutyroyl Hydroxythreonine Hydroxythreonine may be used as a Skin-Conditioning Agents and having other beneficial properties. Palmitoyl Dipeptide-5 Diaminobutyroyl Hydroxythreonine is a preservative free (patented) peptide combination for delivering ingredients to help with the appearance of radiant skin.

In one embodiment, Palmitoyl Dipeptide-5 Diaminobutyrate may be used as a Skin-Conditioning Agents and having other beneficial properties.

In one embodiment, Butyroyl Pentapeptide-24 may be used as a Skin-Conditioning Agents and having other beneficial properties.

In one embodiment, Hydrogenated Lecithin may be used as an Emulsifying Agent and/or Skin-Conditioning Agent. Hydrogenated Lecithin is believed to support healthy and natural skin renewal. The look and appearance of the skin is constantly being affected by the skin cell function. The continuous cell renewal process aids in the look and appearance of a healthy, beautiful complexion. Keeping the skin supported during vital renewal periods is an important step.

In one embodiment, Bidens Pilosa Extract may be used as a Skin-Conditioning Agent and/or a Humectant. Bidens Pilosa Plant Extract may be used for purposes of skin renewal. This botanical plant has been shown clinically to help prevent the formation of early lines and wrinkles Bidens pilosa is an herbaceous plant having retinoid-like activity. Due to the phytochemical composition of Bidens pilosa, especially by the presence of phytochemical compounds supports indications of antioxidant and anti-inflammatory functions and activity on retinoid receptors.

In one embodiment, Mauritia Flexuosa Fruit Oil may be used as a Skin-Conditioning Agent and having other beneficial properties. Mauritia Flexuosa Fruit Oil may be useful in skin hydration. The nutrients found in the Mauritia Flexuosa Fruit Oil helps to provide natural moisturizing and hydrating properties to help skin maintain a smoother and softener look as a result of better hydration and good care. Mauritia Flexuosa Fruit Oil is oil that may be extracted from the Buriti plant or more specifically the Buriti fruit pulp. It is a rich source of carotenoids, tocopherols, vitamin-A, monounsaturated fatty acids, and is known for being a powerful natural antioxidant.

In one embodiment, Elaeis Guineensis (Palm) Oil may be used as a Skin-Conditioning Agent, may be occlusive and have other beneficial properties.

In one embodiment, Gossypium Herbaceum (Cotton) Seed Oil may be used as a Skin-Conditioning Agent and/or Skin-Conditioning Agent—Occlusive.

In one embodiment, Linum Usitatissimum (Linseed) Seed Oil may be used as a Skin-Conditioning Agents and/or Skin-Conditioning Agents—Occlusive.

In one embodiment, Centella Asiatica Leaf Cell Extract may be used as an Antioxidants and/or a Skin Protectant. Centella Asiatica Leaf Cell Extract is believe to provide many nutrients to the skin with phytosterols, amino acids and polysaccharides, making it a unique and nourishing extract. For centuries the Centella Asiatica has also been identified and traditionally used for its anti-inflammatory properties. These qualities help the Centella Asiatica to support healthy skin at the same time it works in the toning and tightening of aging skin. It is further believed it can also contribute to the production of fibroblast, the cells that increase collagen.

In one embodiment, Squalane may be used as a Skin-Conditioning Agents—Occlusive.

In one embodiment, Allantoin may be used as a Skin Protectant, a Skin-Conditioning Agents and may have additional beneficial properties.

In one embodiment, Ubiquinone may be used as an Antioxidant, and/or a Skin-Conditioning Agent. Ubiquinone may have additional beneficial properties. Ubiquinone is also referred to as CoQ-10 and this potent antioxidant is important in assisting the cells in the building of collagen and elastin. Antioxidants are used in cosmetics to help prevent and slow down the signs of aging, as well as for protection and preservation.

In one embodiment, Dipotassium Glcryrrhizinate may be used as a Chelating Agent.

In one embodiment, Xanthan Gum may be used as a Binder, Emulsion Stabilizer, Emulsifying Agent, and/or Viscosity Increasing Agent.

In one embodiment, Disodium EDTA may be used as a Chelating Agent.

In one embodiment, Tocopherol Acetate may be used as an Antioxidant and/or a Skin-Conditioning Agents and may further have other beneficial properties.

In one embodiment, Acrylates/C10-30 Alkyl Acrylate Crosspolymer may be used as an Emulsion Stabilizer and/or a Viscosity Increasing Agent.

In one embodiment, Cetearyl Glucoside may be used as a Skin-Conditioning Agents and/or an Emulsifying Agent and may have additional beneficial properties.

In one embodiment, Phenoxyethanol may be used as a Preservative,

In one embodiment, Sodium Hydroxide may be used as a pH Adjuster.

In one embodiment, Citric Acid may be used as a pH Adjuster.

In one embodiment, natural albumen may be used as a Skin-Conditioning Agent, having other beneficial properties. Natural albumen may help tighten skin. Natural albumen may come in liquid form and is a protein. In use, the natural albumen may help with many signs of aging by aiding tired and stressed skin that often promote the showing of premature lines and wrinkles. The natural albumen may help tighten skin to soften the appearance of fine lines and wrinkles Non-limiting, alternative ingredients may be albumen manufactured by different methods. In certain aspects, a composition may include between about 5% and 15% natural albumen. In other aspect, a composition may include approximately 10% natural albumen. Natural albumin may be used in a number of different compositions. During the compounding process, according to a non-limiting embodiment provided for illustrative purposes only, the natural albumen is kept in a temperature range of about 30 degrees Celsius to about 60 degrees Celsius.

In one embodiment, Oryza Sativa (Rice) Bran Oil may be used as a Skin-Conditioning Agent—Emollient.

In one embodiment, C14-22 Alcohols may be used as Emulsion Stabilizers.

In one embodiment, C12-20 Glucoside may be used as Emulsifying Agents.

In one embodiment, Montanov™ 68, which may be supplied by SEPPIC may be used which may include C14-22 Alcohols and C12-20 Glucoside. The composition of Montanov™ 68 may include approximately 75-85% of C14-22 Alcohols and approximately 15-25% of C12-20 Alkyl Glucoside.

In one embodiment, Cetearyl Alcohol may be used as Emulsion Stabilizers, Opacifying Agents and/or Viscosity Increasing Agents.

In one embodiment, Montanov™ L, which may be supplied by SEPPPIC may be used, which may include approximately 80% Cetearyl Alcohol and 20% Ceary Glucoside.

In one embodiment, Ricinus Communis (Castor) Seed Oil may be used as a Skin-Conditioning Agent—Occlusive.

In one embodiment, Octyldodecyl Olivate may be used as a Skin-Conditioning Agent—Emollient, slip modifier and Occlusive. Octyldodecyl Olivate may help in moisturizing skin. Non-limiting alternatives may include true monoi oil, Emollients like esters, shine ingredients like silicones, Dimethicone, cyclomethicone, etc. This moisturizing ester is derived from the olive tree. For centuries the cosmetic industry has known the benefits and richness the oil from the olive can provide. In certain aspects, a composition may include between about 1% to about 5% of Octyldodecyl Olivate. In other aspects, a composition may include about 3% Octyldodecyl Olivate.

In one embodiment, Olus Oil may be used as a Skin-Conditioning Agent—Emollient.

In one embodiment, Chondrus Crispus Powder may be used as a Viscosity Agent.

In one embodiment, Sodium Isostearoyl Lactylate may be used as an Emulsifying Agent.

In one embodiment, Polysorbate 20 may be used as an Emulsifying Agent and/or a Solubilizing Agent.

In one embodiment, Caprylyl Glycol may be used as a Preservative and/or a Skin Conditioning Agent.

In one embodiment, Cucumis Sativus (Cucumber) Fruit Extract may be used as a Fragrance Ingredient and/or Essential Oil.

In one embodiment, Glyceryl Caprylate may be used as a Skin-Conditioning Agent—Emollient and/or Emulsifying Agent.

In one embodiment, Hydrolyzed Quinoa may be used as a Skin-Conditioning Agent, having additional beneficial properties. Hydrolyzed Quinoa may help repair skin and is known for its essential nutrients. It is believed Hydrolyzed Quinoa assists in the repair, protection and conditioning of the skin. In certain aspects, a composition may include between about 0.05% to about 0.2 percent of Hydrolyzed Quinoa. In other aspects, a composition may include about 0.10% of hydrolyzed quinoa.

In one embodiment, Phenylpropanol may be used as a preservative.

In one embodiment Dermasoft™ LP which may be supplied by Prospector may be used and may contain Caprylyl Glycol, Glycerin and Glyceryl Caprylate, Phenylpropanol and Water. Dermasoft™ LP may include approximately 50% Caprylyl Glycol, 5-10% Glycerin and Glyceryl Caprylate, 5-10% Phenylpropanol and 1-5% Water.

In one embodiment, Sodium PCA may be used as a Humectant and/or Skin-Conditioning Agent.

In one embodiment, Stearic Acid may be used as an Emulsifying Agent.

In one embodiment, Tocopherol may be used as an Antioxidant, a Skin-Conditioning Agent, and may be used for additional known beneficial properties. Tocopherol may also be Occlusive. Tocopherol may also be known as Vitamin E and has been used for generations for its moisturizing, nourishing and protection properties. When used topically it aids and assists in helping to provide an antioxidant defense for the skin. It is a fat-soluble antioxidant.

II. Compositions/Compounds

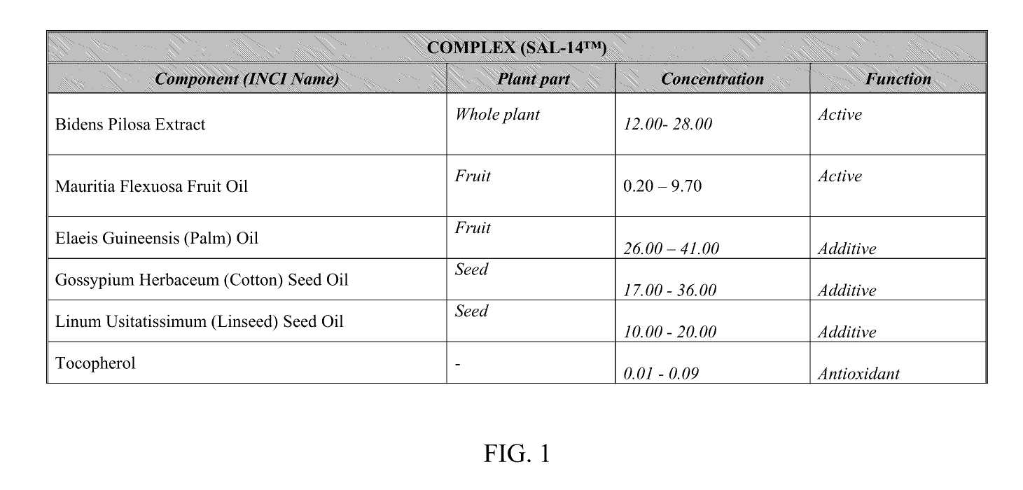

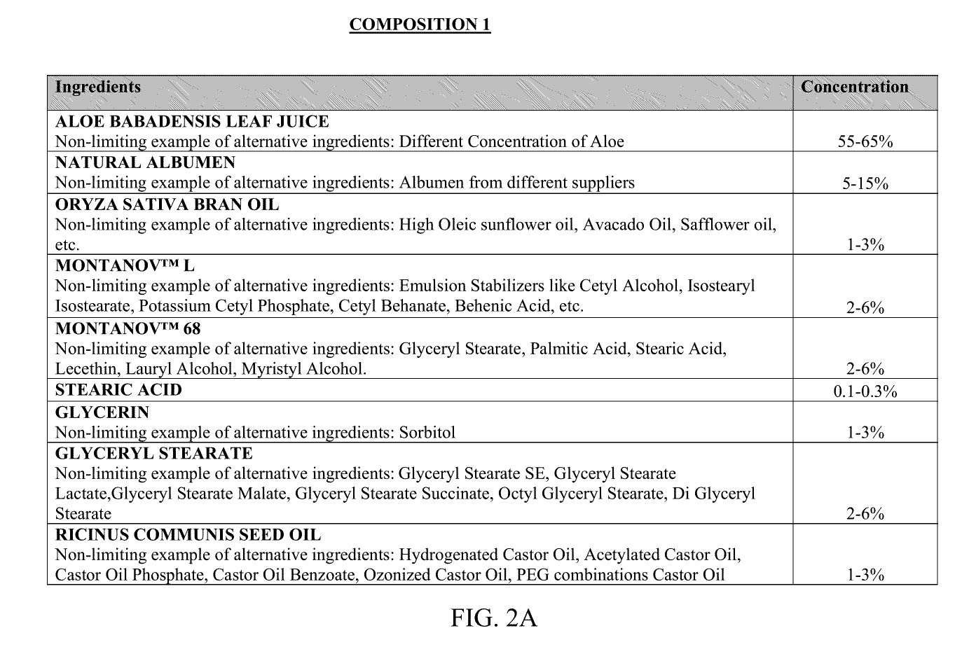

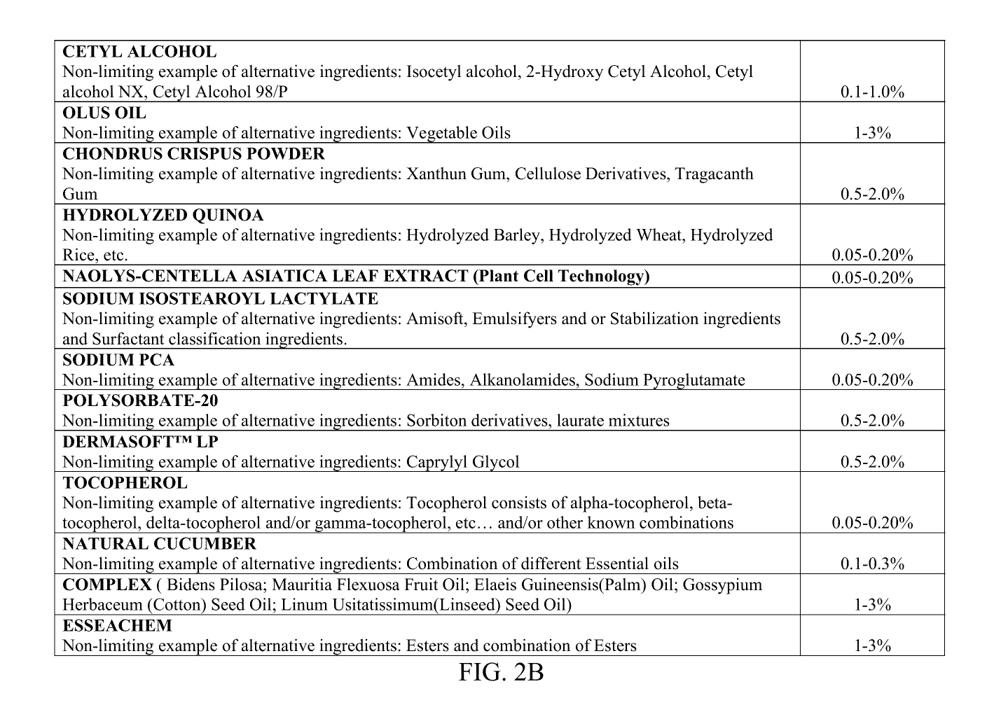

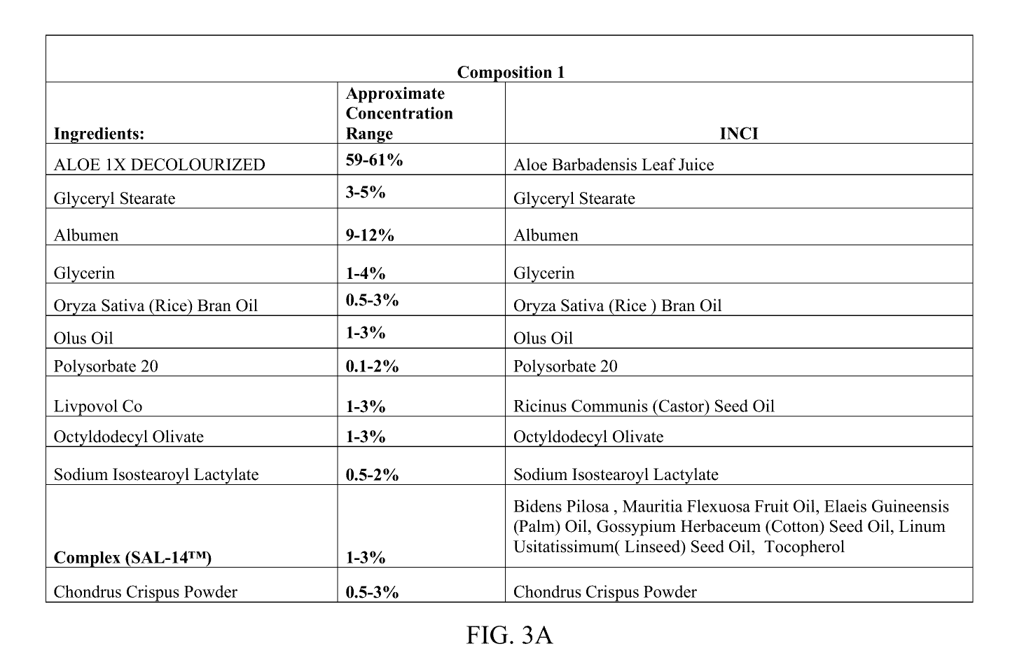

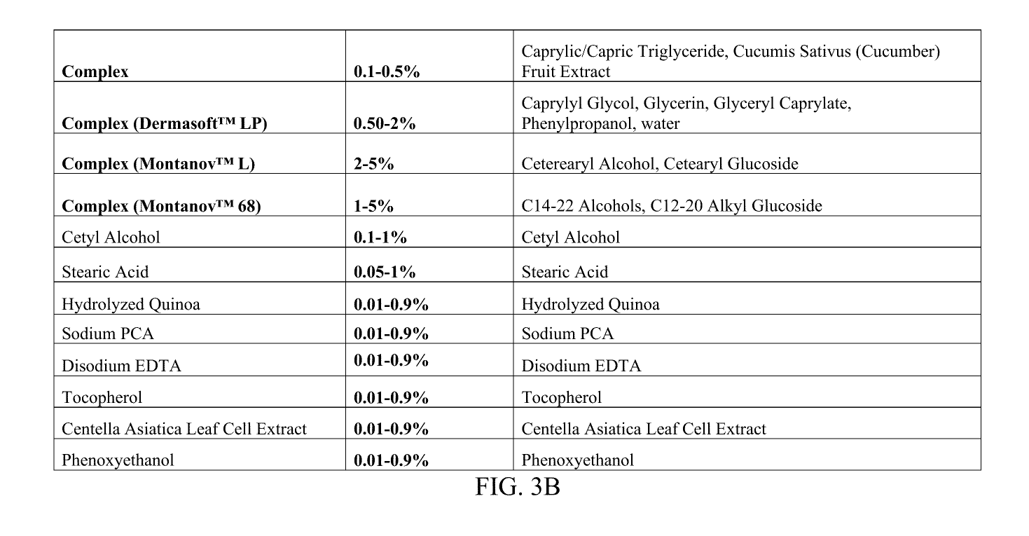

Referring to FIG. 1, an illustrative composition or complex is presented. The complex is a genotoxic compound or at least a DNA composition having protective capacities for cosmetic and pharmaceutical use after sun-induced stress. The complex includes Bidens Pilosa Extract in a concentration of approximately 12-28%, Mauritia Flexuosa Fruit Oil in a concentration of approximately 0.2-10%, Elaeis Guineensis (Palm) Oil in a concentration of about 26-41%, Gossypium Herbaceum (Cotton) Seed Oil in a concentration of about 17-36%, Linum Usitatissimum (Linseed) Seed Oil in a concentration of about 10-20%, and Tocopherol in a concentratio of about 0.01-0.09%. In some aspects, the complex includes Bidens Pilosa Extract in a concentration of approximately 20-28%, Mauritia Flexuosa Fruit Oil in a concentration of approximately 5-10%, Elaeis Guineensis (Palm) Oil in a concentration of about 26-35%, Gossypium Herbaceum (Cotton) Seed Oil in a concentration of about 17-34%, Linum Usitatissimum (Linseed) Seed Oil in a concentration of about 10-15%, and Tocopherol in a concentratio of about 0.01-0.05%. As illustrated in FIGS. 2-3, the complex of FIG. 1 may only comprise, in a non-limiting embodiment, about 2% of the respective compositions illustrated therein. The complex of FIG. 1 may, in some aspects comprise about 1-5% of the composition.

Referring to FIGS. 2-3B, non-limiting, illustrative embodiments of a composition, which may be used in the morning or at night, but preferably at night, includes a combination, either all or in part, according to ingredient and the contributing percentage of each ingredient. It will be appreciated to one of ordinary skill that the percentages are approximations and are provided for purposes of example only and that the percentages may vary and substitute ingredients noted, and known in the art, may be used. Likewise, the composition may use one or more of the ingredients listed. The composition does not have to use all of the ingredients listed. The following composition includes Aloe Barbadensis Leaf Juice, Natural Albumen, Glyceryl Stearate, Oryza Sativa (Rice) Bran Oil, C14-22 Alcohols, C12-20 Glucoside, Cetearyl Alcohol, Cetearyl Glucoside, Ricinus Communis (Castor) Seed Oil, Octyldodecyl Olivate, Olus Oil, Glycerin, Chondrus Crispus Powder, Bidens Pilosa Extract, Mauritia Flexuosa Fruit Oil, Elaeis Guineensis (Palm) Oil, Gossypium Herbaceum (Cotton) Seed Oil, Linum Usitatissimum (Linseed) Seed Oil, Cetyl Alcohol, Stearic Acid, Centella Asiatica Leaf Cell Extract, Sodium PCA, Sodium Isostearoyl Lactylate, Polysorbate 20, Caprylyl Glycol, Glyceryl Caprylate, Phenylpropanol, Hydrolyzed Quinoa, Tocopherol, Caprylic/Capric Triglyceride, Cucumis Sativus (Cucumber) Fruit Extract. It will be appreciated to one of skill in the art that known alternatives to the above ingredients may be used in substitution.

In one embodiment, approximate concentrations of the following ingredients may be utilized to blend the composition: Aloe Barbadensis Leaf Juice—60%, Natural Albumen—10%, Glyceryl Stearate—4%, Oryza Sativa (Rice) Bran Oil—2%, C14-22 Alcohols and C12-20 Glucoside—4%, Cetearyl Alcohol and Cetearyl Glucoside—4%, Ricinus Communis (Castor) Seed Oil—2%, Octyldodecyl Olivate—2%, Olus Oil—2%, Glycerin—2%, Chondrus Crispus Powder—1%, Bidens Pilosa Extract and Mauritia Flexuosa Fruit Oil and Elaeis Guineensis (Palm) Oil and Gossypium Herbaceum (Cotton) Seed Oil and Linum Usitatissimum (Linseed) Seed Oil and Tocopherol—2%, Cetyl Alcohol—0.5%, Stearic Acid—0.2%, Centella Asiatica Leaf Cell Extract—0.1%, Sodium PCA—0.1%, Sodium Isostearoyl Lactylate—1%, Polysorbate 20—1%, Caprylyl Glycol and Glyceryl Caprylate and Phenylpropanol—1.5%, Hydrolyzed Quinoa —0.1%, Tocopherol—0.1%, Caprylic/Capric Triglyceride and Cucumis Sativus (Cucumber) Fruit Extract—0.2%.

A composition, according to one embodiment, that may be used either in the morning or at night, but preferably at night, includes main ingredients comprising bidens pilosa plant extract and centella asiatica leaf cell extract. In one embodiment, the composition further includes mauritia flexuosa fruit oil as a main ingredient and Caprylic/Capric Triglyceride and Cucumis Sativus (Cucumber) Fruit Extract. The composition further includes any combination of the following: Aloe Barbadensis Leaf Juice, Natural Albumen, Octyldodecyl Olivate, Hydrolyzed Quinoa, Glycerin, Glyceryl Caprylate, Phenylpropanol, and/or Tocopherol. It will be appreciated to one of skill in the art that known alternatives to the above ingredients may be used in substitution.

A composition, according to one embodiment, that may be used either in the morning or at night, but preferably at night, includes main ingredients comprising extract of bidens pilosa; mauritia flexuosa fruit oil; jojoba esters; centella asiatica leaf cell extract; and cucumis sativus fruit extract.

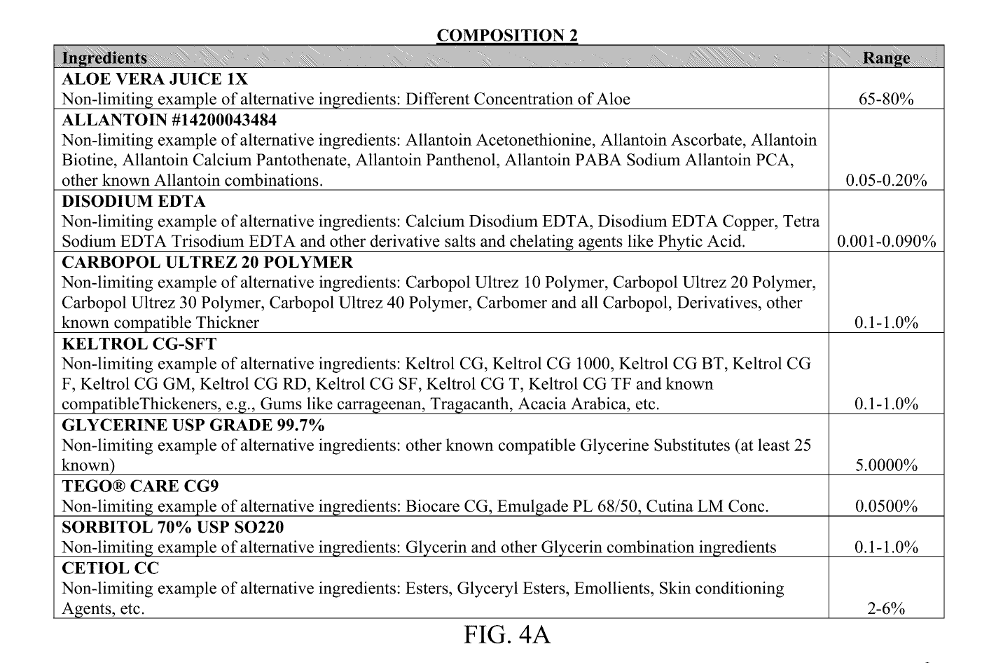

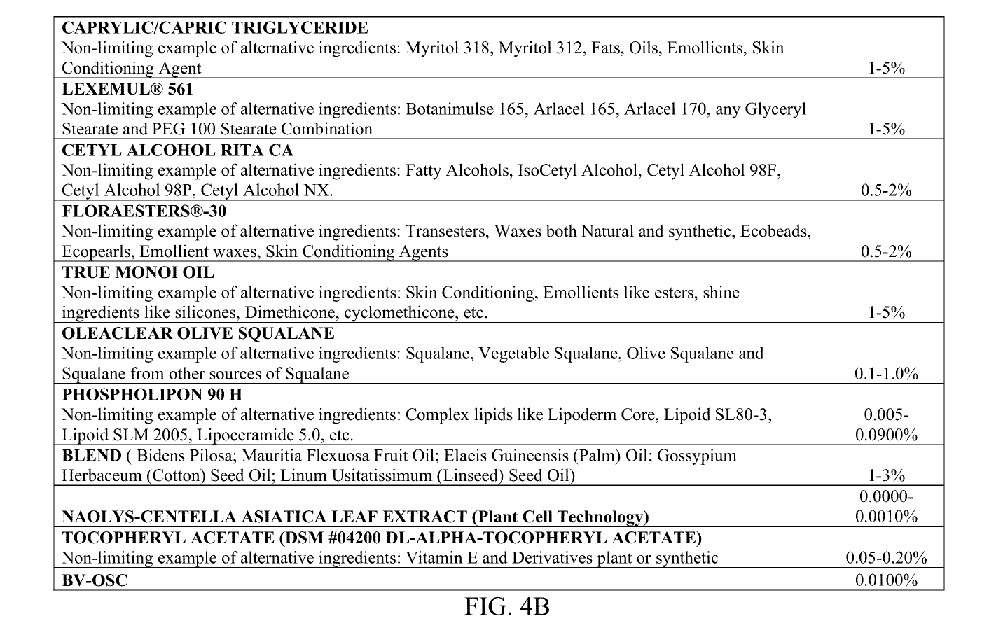

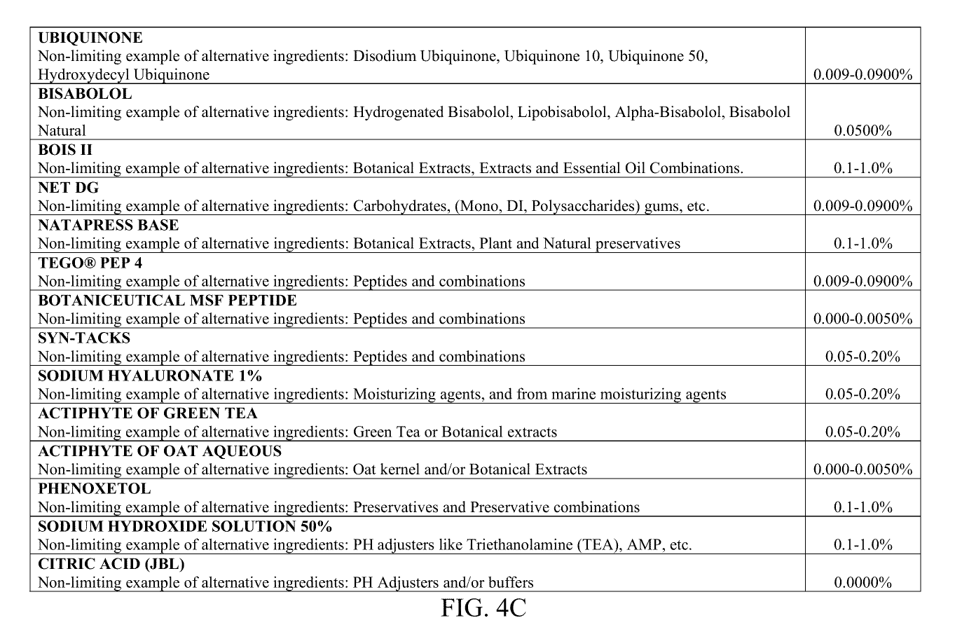

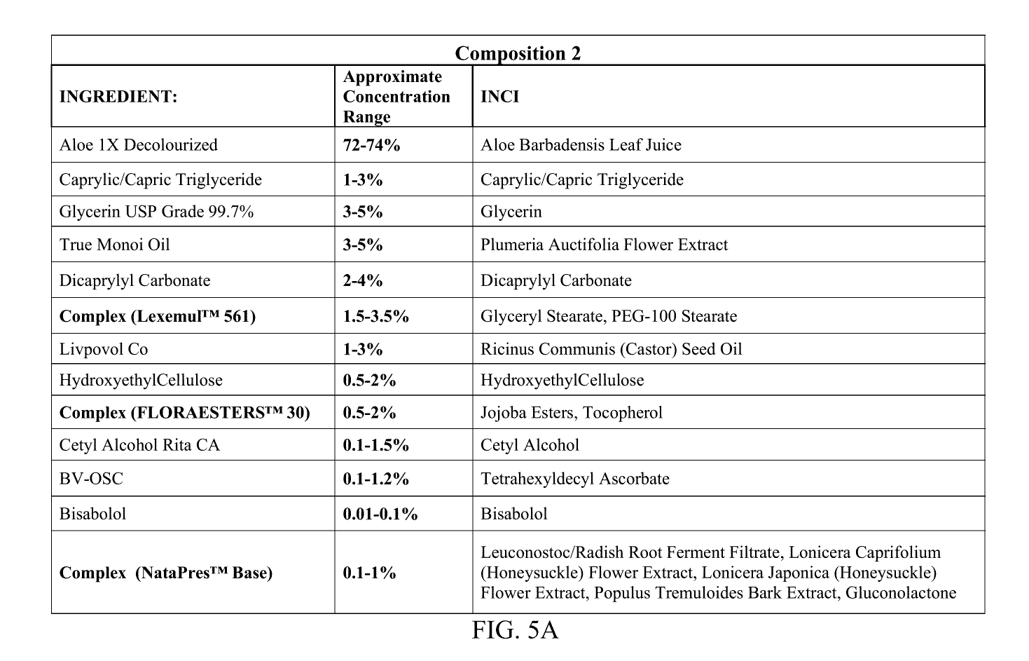

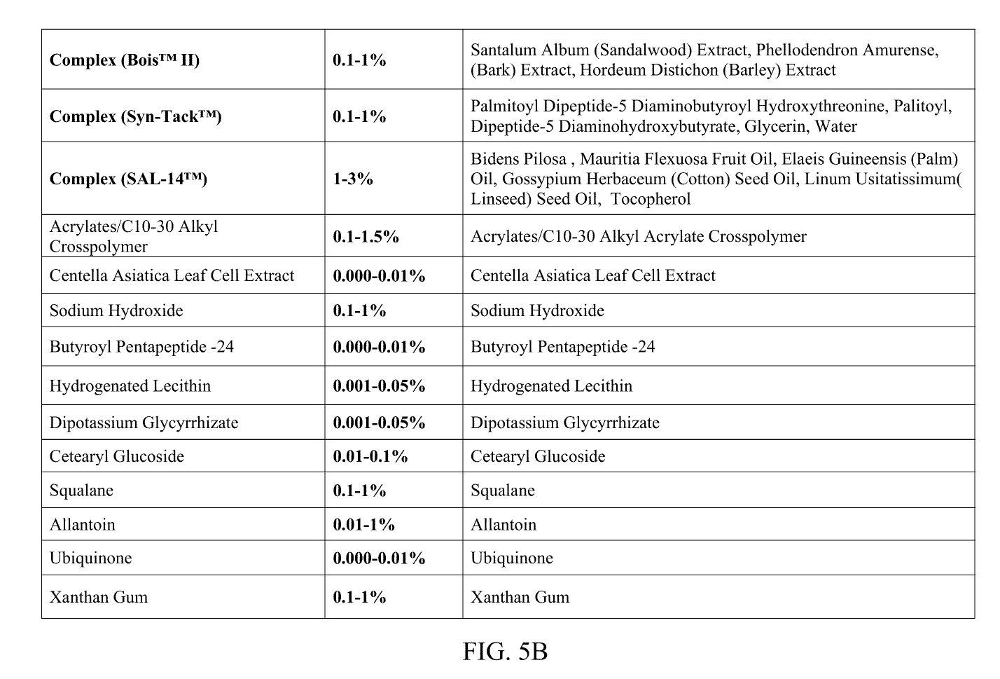

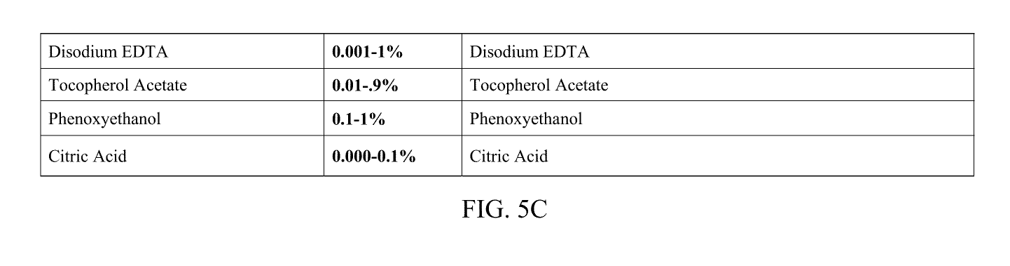

FIGS. 4-5C illustrate exemplary, non-limiting embodiments, of a composition that may be used either in the morning or at night, but preferably in the morning, that includes a combination, either all or in part, according to ingredient and the contributing percentage of each ingredient. It will be appreciated to one of ordinary skill that the percentages are approximations and are provided for purposes of example only and that the percentages may vary and substitute ingredients noted, and known in the art, may be used. Likewise, the composition may use one or more of the ingredients listed. The composition does not have to use all of the ingredients listed. The following composition includes a combination, either all or in part, of Aloe Barbadensis Leaf Juice, Caprylic/Capric Triglyceride, Glycerin, Plumeria Auctifolia Flower Extract, Dicaprylyl Carbonate, Complex (Glyceryl Stearate and PEG-100 Stearate), Ricinus Communis (Castor) Seed Oil, HydroxyethylCellulose, Complex (Jojoba Esters and Tocopherol), Cetyl Alcohol, Tetrahexyldecyl Ascorbate, Bisabolol, Complex (Leuconostoc/Radish Root Ferment Filtrate and Lonicera Caprifolium (Honeysuckle) Flower Extract and Lonicera Japonica (Honeysuckle) Flower Extract and Populus Tremuloides Bark Extract and Gluconolactone), Complex (Santalum Album (Sandalwood) Extract and Phellodendron Amurense and (Bark) Extract and Hordeum Distichon (Barley) Extract), Complex (Palmitoyl Dipeptide-5 Diaminobutyroyl Hydroxythreonine and Palitoyl, Dipeptide-5 Diaminohydroxybutyrate and Glycerin and Water), Complex (Bidens Pilosa and Mauritia Flexuosa Fruit Oil and Elaeis Guineensis (Palm) Oil and Gossypium Herbaceum (Cotton) Seed Oil and Linum Usitatissimum(Linseed) Seed Oil and Tocopherol), Acrylates/C10-30 Alkyl Acrylate Crosspolymer, Centella Asiatica Leaf Cell Extract, Sodium Hydroxide, Butyroyl Pentapeptide-24, Hydrogenated Lecithin, Dipotassium Glycyrrhizate, Cetearyl Glucoside, Squalane, Allantoin, Ubiquinone, Xanthan Gum, Disodium EDTA, Tocopherol Acetate, Phenoxyethanol. It will be appreciated to one of skill in the art that known alternatives to the above ingredients may be used in substitution.

In one non-limiting embodiment the composition for preferable use in the morning includes one or more of the following ingredients having approximate percentages: Aloe Barbadensis Leaf Juice—73%, Caprylic/Capric Triglyceride—2%, Glycerin—4%, Plumeria Auctifolia Flower Extract—4%, Dicaprylyl Carbonate—3%, Glyceryl Stearate and PEG-100 Stearate—2.5%, Ricinus Communis (Castor) Seed Oil—2%, HydroxyethylCellulose—1%, Jojoba Esters and Tocopherol 1%, Cetyl Alcohol—0.8%, Tetrahexyldecyl Ascorbate—0.5%, Bisabolol—0.05%, Complex (Leuconostoc/Radish Root Ferment Filtrate and Lonicera Caprifolium (Honeysuckle) Flower Extract and Lonicera Japonica (Honeysuckle) Flower Extract and Populus Tremuloides Bark Extract and Gluconolactone)—0.5%, Complex (Santalum Album (Sandalwood) Extract and Phellodendron Amurense and (Bark) Extract and Hordeum Distichon (Barley) Extract)—0.5%, Complex (Palmitoyl Dipeptide-5 Diaminobutyroyl Hydroxythreonine and Palitoyl, Dipeptide-5 Diaminohydroxybutyrate and Glycerin and Water)—0.25%, Complex (Bidens Pilosa and Mauritia Flexuosa Fruit Oil and Elaeis Guineensis (Palm) Oil and Gossypium Herbaceum (Cotton) Seed Oil and Linum Usitatissimum(Linseed) Seed Oil and Tocopherol)—2%, Acrylates/C10-30 Alkyl Acrylate Crosspolymer—0.6%, Centella Asiatica Leaf Cell Extract—0.001%, Sodium Hydroxide—0.5%, Butyroyl Pentapeptide-24—0.001%, Hydrogenated Lecithin—0.01%, Dipotassium Glycyrrhizate—0.01%, Cetearyl Glucoside—0.05%, Squalane—0.5%, Allantoin—0.1%, Ubiquinone—0.001%, Xanthan Gum—0.5%, Disodium EDTA—0.01%, Tocopherol Acetate—0.01%, Phenoxyethanol—0.5%.

A composition, according to one embodiment, that may be used either in the morning or at night, but preferably in the morning, includes main ingredients comprising bidens pilosa plant extract and centella asiatica leaf cell extract. In one embodiment, the composition further includes mauritia flexuosa fruit oil as a main ingredient. The composition further includes any combination of the following: Jojoba Esters, Camellia Sinensis (green Tea) Leaf Extract, Hydrogenated Lecithin, Ubiquinone, Bisabolol, Terapeptide-30 and Palmitoyl dipeptide-5 diaminobutyroyl hydroxythreonine. It will be appreciated to one of skill in the art that known alternatives to the above ingredients may be used in substitution.

A composition, according to one embodiment, that may be used either in the morning or at night, but preferably in the morning, includes main ingredients comprising bidens pilosa plant extract and centella asiatica leaf cell extract. In one embodiment, the composition further includes mauritia flexuosa fruit oil as a main ingredient. The composition may further include Tetrahexyldecyl Ascorbate, Jojoba Esters, and Tocopherol. It will be appreciated to one of skill in the art that known alternatives to the above ingredients may be used in substitution.

In the foregoing description of certain embodiments, specific terminology has been resorted to for the sake of clarity. The disclosure, however, is not intended to be limited to the specific terms so selected, and it is to be understood that each specific term includes other technical equivalents that operate in a similar manner to accomplish a similar technical purpose.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

In addition, the foregoing describes only some embodiments of the invention(s), and alterations, modifications, additions and/or changes can be made thereto without departing from the scope and spirit of the disclosed embodiments, the embodiments being illustrative and not restrictive.

Furthermore, invention(s) have described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention(s). Also, the various embodiments described above may be implemented in conjunction with other embodiments, e.g., aspects of one embodiment may be combined with aspects of another embodiment to realize yet other embodiments. Further, each independent feature or component of any given assembly may constitute an additional embodiment.

What is claimed is:

1. A topical composition comprising:
(a) extract of bidens pilosa;
(b) centella asiatica leaf cell extract; and
(c) a dermatologically acceptable carrier.

2. The topical composition of claim 1 further comprising mauritia flexuosa fruit oil.

3. The topical composition of claim 1 further comprising elaeis guineessis oil.

4. The topical composition of claim 1 further comprising gossypium herbaceum seed oil.

5. The topical composition of claim 1 further comprising linum usitatissimum seed oil.

6. The topical composition of claim 1 further comprising tocopherol.

7. The topical composition of claim 1 further comprising aloe barbadensis leaf juice, natural albumen, octyldodecyl olivate, hydrolyzed quinoa, and tocopherol.

8. The topical composition of claim 1 further comprising jojoba esters, camellia sinensis leaf extract, hydrogenated lecithin, ubiquinone, bisabolol, terapeptide-30, and palmitoyl dipeptide-5 diaminobutyroyl hydroxythreonine.

9. A method for treating an area of skin to reduce DNA fragmentation induced by ultra-violet radiation comprising topically applying a composition to the area of skin, the composition comprising extract of bidens pilosa and centella asiatica leaf cell extract.

10. The method of claim 9, wherein the composition further comprises mauritia flexuosa fruit oil.

11. The method of claim 9, wherein the composition further comprises aloe barbadensis leaf juice, natural albumen, octyldodecyl olivate, hydrolyzed quinoa, and tocopherol.

12. The method of claim 11, wherein the composition further comprises oryza sativa bran oil, montanov L, montanov 68, stearic acid, glycerin, glyceryl stearate, ricinus communis seed oil, cetyl alcohol, olus oil, chondrus crispus powder, sodium isostearoyl lactylate, sodium PCA, polysorbate-20, dermsoft LP, natural cucumber, and esseachem.

13. The method of claim 9, wherein the composition further comprises jojoba esters, camellia sinensis leaf extract, hydrogenated lecithin, ubiquinone, bisabolol, terapeptide-30, and palmitoyl dipeptide-5 diaminobutyroyl hydroxythreonine.

14. The method of claim 11, wherein the composition further comprises aloe vera juice.

15. The method of claim 9 further including cleansing the area of the skin prior to applying the composition.

16. The method of claim 9 further including applying the composition once daily in the morning.

17. The method of claim 9 further including applying the composition once daily in the evening.

18. The method of claim 9 further including applying the composition once in the morning and once in the evening.

19. The method of claim 9 further including applying the composition as needed for moisturizing the area of skin.

20. The method of claim 9 further used to treat the area of skin by reducing appearance of fine lines and wrinkles, discoloration, uneven texture, enlarged pores, aging or loose skin.

21. A topical composition comprising:
(a) extract of bidens pilosa;
(b) mauritia flexuosa fruit oil;
(c) jojoba esters;
(d) centella asiatica leaf cell extract; and
(e) cucumis sativus fruit extract.

22. A topical composition comprising:
(a) extract of bidens pilosa;
(b) mauritia flexuosa fruit oil;
(c) jojoba esters;
(d) centella asiatica leaf cell extract; and
(e) tetrahexyldecyl ascorbate.
